(19) United States
(12) Patent Application Publication
(10) Pub. No.: US 2022/0288370 A1
(43) Pub. Date: Sep. 15, 2022

(54) Method of Manufacturing Microstructures

(71) Applicant: Innoture IP Limited, London (GB)

(72) Inventors: Ryan Bamsey, London (GB); Ionut Cameliu Ichim, London (GB)

(73) Assignee: Innoture IP Limited, London (GB)

(21) Appl. No.: 17/636,320

(22) PCT Filed: Aug. 20, 2020
(86) PCT No.: PCT/GB2020/052007

§ 371 Date: Feb. 17, 2022

(30) Foreign Application Priority Data
Aug. 20, 2019 (GB) 1911909.8

(51) Int. Cl. A61M 37/00; B29C 67/08

(52) U.S. Cl. CPC A61M 37/0015; B29C 67/08; A61M 2037/0053; A61M 2037/0023; A61M 2037/0061; B29L 2031/756

(57) ABSTRACT

The present invention provides a novel method for manufacturing a microstructure via the use of a deep template, particularly microstructures that may be found on medical devices, such as transdermal patches, for either cosmetic or medicinal purposes.

### FIELD OF THE INVENTION

The present invention relates to a novel method of manufacturing microstructures, particularly microstructures that may be found on medical devices, such as transdermal patches, for either cosmetic or medicinal purposes.

### BACKGROUND

Transdermal drug delivery has long been regarded as an important route for the administration of active pharmaceutical ingredients, partly due to the rise in the number of drugs which are unsuitable for oral administration due to extensive processing in the gut and liver or susceptibility to the acidic conditions of the stomach. Additionally, transdermal drug delivery offers an alternative delivery system to more invasive approaches and gives the patient a higher degree of freedom compared to methods requiring the presence of a medical professional to administer such therapies, such as parenteral administration. While it is possible to transdermally administer certain drugs, for example by applying a gel or coated patch to the skin, the outermost layer of the skin, a barrier of 10-20 μm termed the stratum corneum, which evolved to prevent the unwanted entry of micro-organisms and toxic substances, also prevents, or at least significantly reduces, the entry of most pharmaceutical compounds.

As a result, a number of methods have been devised to aid delivery of pharmaceutical compounds across the stratum corneum. One means is the use of patches comprising microstructures, specifically microneedles, which are capable of achieving the aforementioned. Due to their size, microneedles have the added advantage over conventional hypodermic needles in that they may only penetrate the epidermis, and potentially not come into contact with sensory neurones located deeper in the skin. Microneedles may reduce or avoid the pain and compliance issues often associated with injections. Previous transdermal patches have been manufactured using various metals and hard plastics, however these patches lack the flexibility required to adapt to different areas of the body and cannot sustain the addition of large numbers of microstructures across a large surface area.

The inventors have previously described an improved method for producing microstructures, in particular microneedles (outlined in EP1786580 and

U.S. Pat. No. 8,192,787), wherein existing polymer and microprocessor technologies have been adapted to produce a superior method of lithographic deposition of polymers. This method uses a ‘layering technique’, involving the use of templates and squeegee blades, in which different microstructure designs can be created. For example, various needle sizes and shapes can be produced, the final product of which is suitable for various flexible substrates over large surface areas. Flexibility is important when designing transdermal patches, for example, which need to be able to flexibly adapt to the contours of the human body.

The present invention provides a means to improve and expand the uses of these known patches containing microstructures by providing a novel and surprisingly advantageous method of manufacturing the microstructures. This novel manufacturing method enables a more efficient, more accurate and more cost effective process of producing microstructures. The present invention is useful for manufacturing microstructures on any substrate, not just limited to transdermal patches.

Previous methods of manufacturing a microstructure have used templates, or ‘stencils’, for depositing the microstructure composition upon the substrate at the desired location. The templates used for the above purpose are known to have a depth of between 10 and 250 μm. The templates currently used in this process are known to create a number of issues that the current inventors have endeavoured to overcome. Firstly, current methods of manufacture use a blend of contact printing and non-contact printing to create a space between the template and the substrate, often leading to issues with the reliability of the image being printed across the substrate, for example, edges of the microstructure not being printed or resulting in an uneven result. Secondly, current templates allow for the flexing of the template when the squeegee blade manipulates the microstructure composition across it, resulting in inaccuracies between the microstructures created on the same substrate. This could lead to issues with particular end applications. For example, if the microstructure was intended to deliver a certain amount of drug to a subject, the lack of uniformity across the microstructures may lead to inaccuracies in drug dosing. Thirdly, the templates currently used in this process often remain attached to the substrate after a print cycle. Upon disengagement of the template from the substrate, it is common for the substrate to be shifted and as a result be irreversibly damaged. Finally, current templates used for the manufacture of microstructures are limited to how much volume of microstructure composition they can contain. Often this leads to multiple printing cycles required, leading to a time consuming and energy inefficient process.

The inventors have identified a novel method of manufacturing microstructures that has a high speed of operation, high accuracy, high reproducibility and the scalability for industrial manufacture that previously used methods were unable to achieve.

The listing or discussion of an apparently prior-published document in this specification should not necessarily be taken as an acknowledgment that the document is part of the state of the art or is common general knowledge.

### SUMMARY OF THE INVENTION

The present invention is based on the surprising discovery by the inventors that the manufacture of a microstructure using a perforated template, with a depth of at least 300 μm, results in a more time efficient and accurate process compared to previously used templates.

Accordingly, the present invention provides a method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template and/or the through-hole have a depth of at least 300 μm.

The present invention also provides a method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template has a rigidity configured to resist deformation.

The method may further comprise the step of exposing the microstructure composition deposited on the substrate to a curing agent, preferably wherein the curing agent is ultraviolet (UV) light, and may comprise one or more further steps comprising repeating the application of the microstructure composition, optionally wherein the perforated template is moved away from the substrate and re-positioned and aligned, such that the through-hole aligns with the microstructure, between each application of the microstructure composition.

The invention also provides a transdermal patch comprising microstructures manufactured using the methods of the invention. The invention also provides a kit comprising a transdermal patch according to the invention and instructions for use of the patch.

The present invention further provides for a therapeutic or cosmetic method of treating a condition in a patient in need thereof comprising applying the transdermal patch herein described to an exposed surface on a subject.

The invention will now be described in more detail with reference to the following figures and examples

### DESCRIPTION OF FIGURES

FIG. 1: A schematic showing the printing process with a thinner template resulting in deflection issues due to higher pressures and space between the stencil and the substrate.

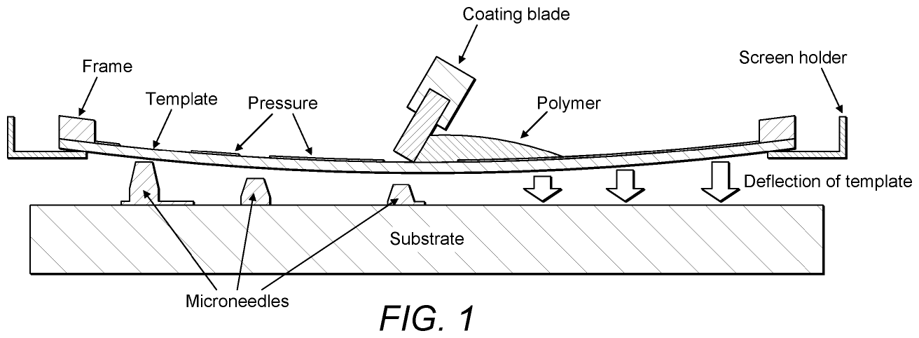

FIG. 2: A schematic showing the printing process with a deeper template resulting in higher pressures being used, use of more viscous polymers and a reduction in template deflection.

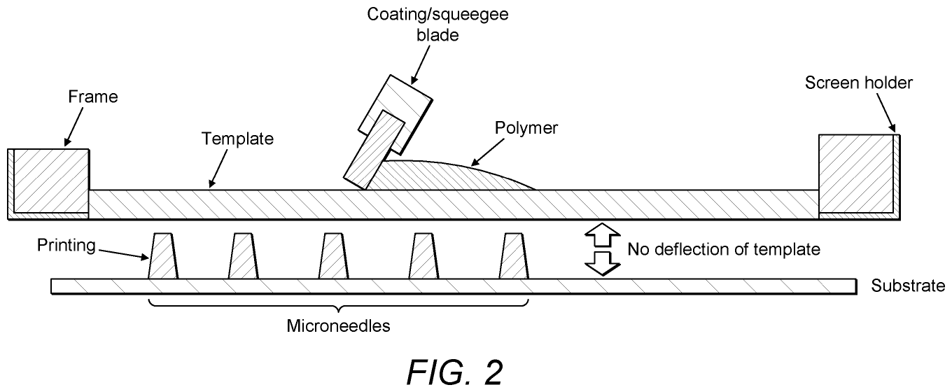

FIG. 3: A schematic demonstrating how the use of a deeper template allows for contact printing throughout the process by removing template deflection completely.

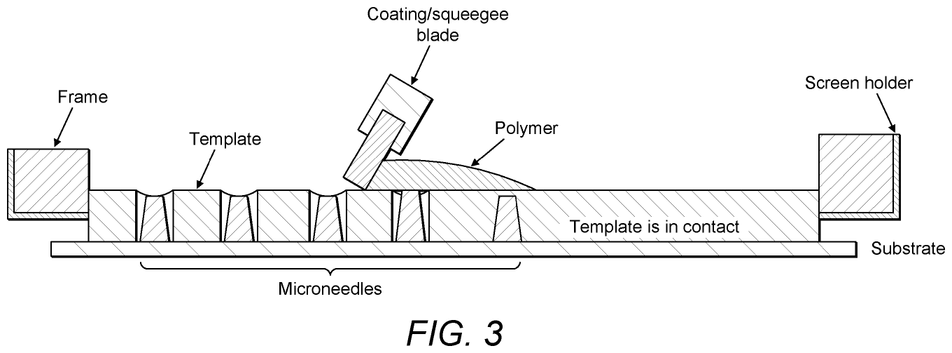

FIG. 4: An example micrograph of a microstructure built using a 500 μm deep template in 5 passes.

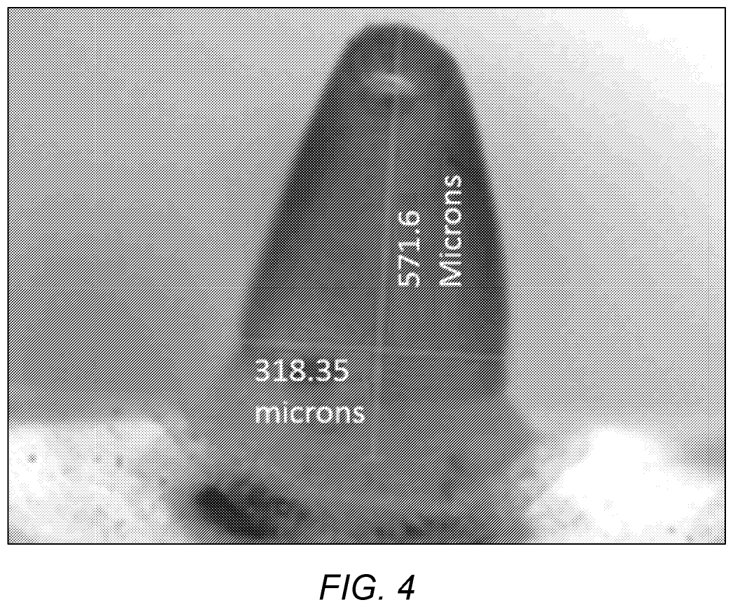

FIG. 5: An example micrograph of a microstructure which has been deformed as a result of template deflection.

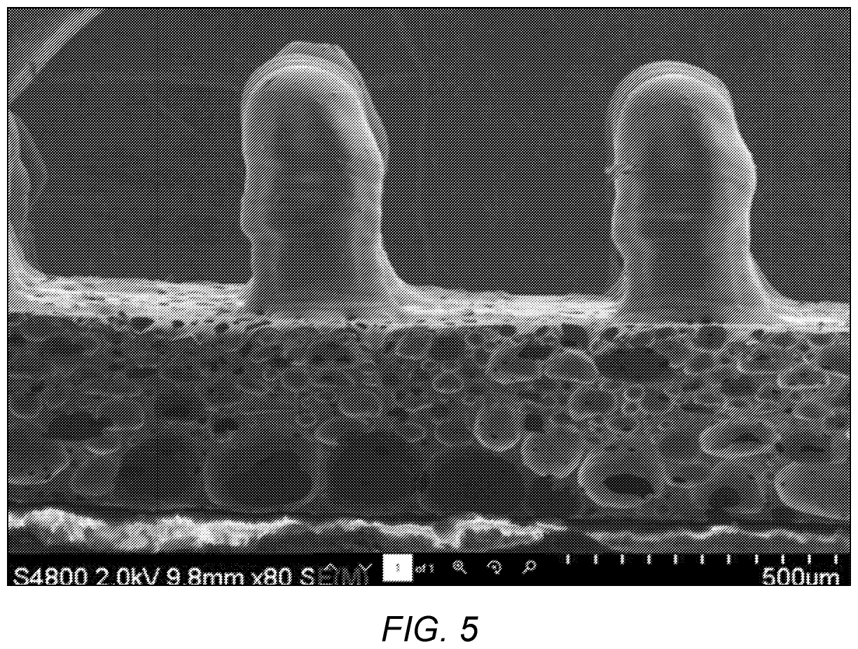

FIG. 6: An example micrograph of microstructures that have been printed unevenly as a result of template deflection.

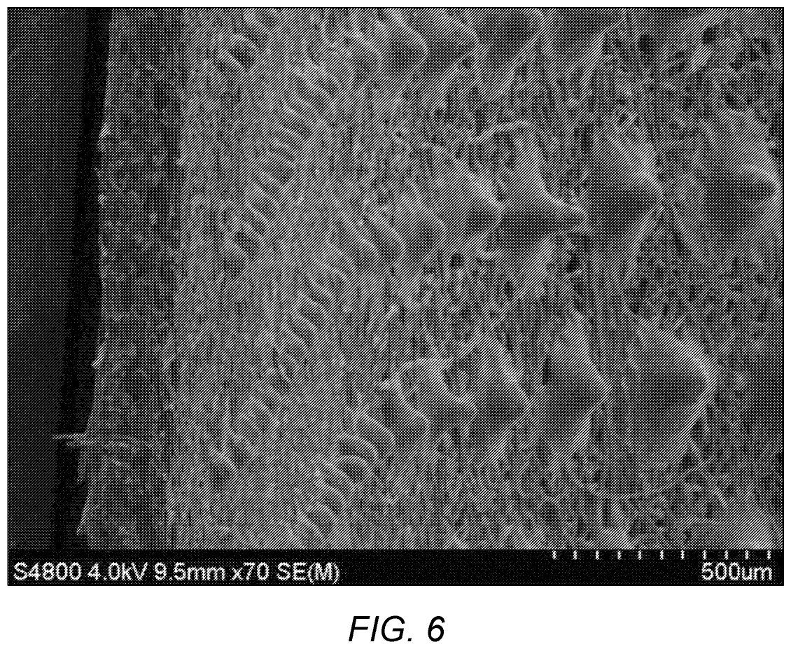

FIG. 7: An example micrograph displaying inaccurate deposition of a polymer due to non-contact printing.

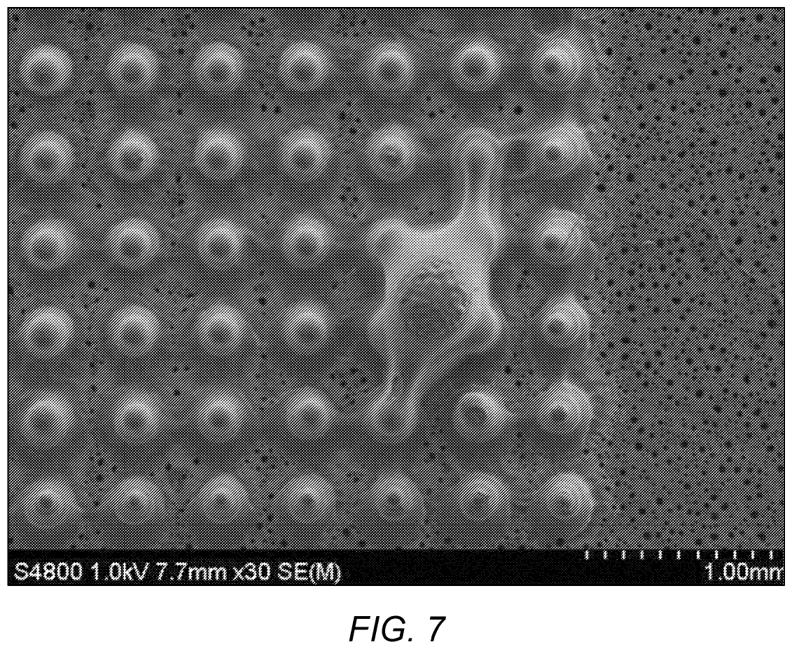

FIG. 8: An example micrograph showing non-build of microstructures due to stencil deflection under higher pressures.

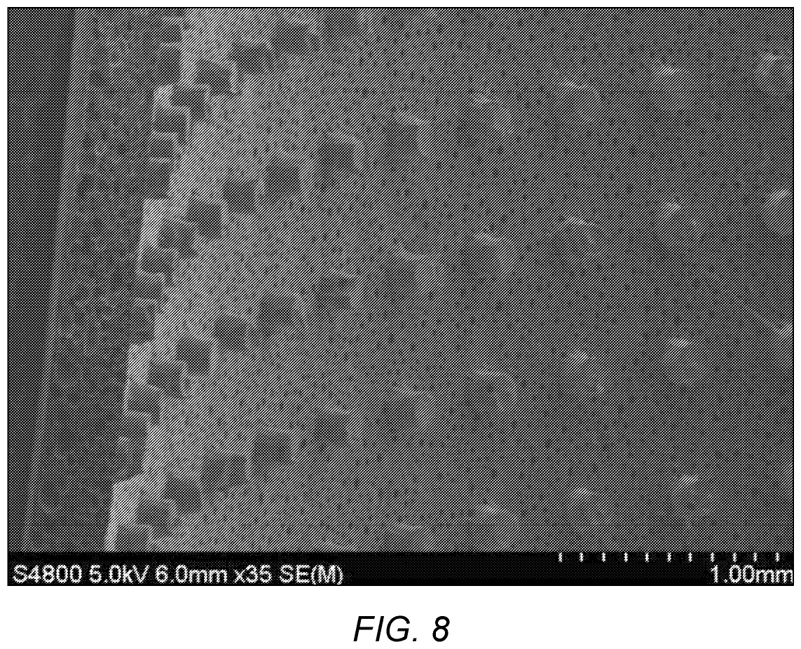

### DETAILED DESCRIPTION

The following description is presented to enable any person skilled in the art to make and use the invention. Various modifications to the disclosed embodiments will be readily apparent to those skilled in the art.

The present invention provides a novel method by which microstructures may be accurately and efficiently manufactured via the use of a perforated template with ‘deep’ through-holes or the use of a perforated template which has a rigidity configured to resist deformation. A number of surprising advantages are associated with this approach compared to using a perforated template with shallower through-holes throughout. Firstly, the use of a deeper/thicker template allows for 100% contact printing if desired, whilst protecting the developing microstructure being built inside the through-hole at the same time. Secondly, the deeper template resists flexing more effectively than templates of a shallower depth when a squeegee blade (for depositing the microstructure composition) moves across the template. Thirdly, surprisingly, due to the reduction in flex of the deeper template, there is a significant reduction in the amount of substrate to be cured which sticks to the template. Sticking can cause movement and damage to the newly built microstructure. Finally, the use of a deeper template provides a more efficient process that enables deposition of a larger volume of microstructure composition than a template with a shallower depth, therefore requiring less cycles of deposition and producing a finalised product more quickly. Accordingly, the present invention solves the problems associated with previously used methods known in the prior art.

In order to place the present invention in the appropriate context, and provide familiarisation with the terms used throughout this application, we have provided a brief overview of the previously used method for manufacturing microstructures according to EP1786580 and U.S. Pat. No. 8,192,787. The present invention provides significant advantages over this method. Briefly, the substrate to which the desired microstructure composition is to be deposited is aligned with a ‘perforated template’ or ‘stencil’ containing numerous ‘apertures’ or ‘through-holes’ using the appropriate technology. The microstructure composition is applied to the ‘perforated template’ or ‘stencil’ and subsequently urged through the ‘through-holes’ or ‘apertures’ and the desired amount of the microstructure composition is deposited onto the receiving substrate. The amount of microstructure composition that is deposited onto the receiving substrate on each pass is referred to as the ‘deposit yield’. To urge the microstructure composition through the ‘perforated template’ or ‘stencil’, a squeegee blade, or equivalent, is used to direct the microstructure composition to the desired position, and urged through the through-holes of the perforated template, by applying pressure to the template. The above terms will be used throughout the following description of the present invention. Two methods of printing may be employed; 1) non-contact printing, where the template is not in contact with the substrate and 2) contact printing, where the template is in contact with the substrate.

Accordingly, in a first aspect the present invention provides a method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template and/or the through-hole have a depth of at least 300 μm.

The present invention also provides a method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template has a rigidity configured to resist deformation. In a preferred embodiment, the perforated template resists deformation of a print pressure of, or from, at least 1 kg to 20 kg.

The term ‘rigidity’ is intended to refer to the stiffness or the tensile strength of the perforated template. In the context of the present invention, these terms refer to the improved ability of the perforated template to not be bent, forced out of shape, or deformed when a print pressure (for example from the application of the squeegee blade), previously known to result in deformation, is applied.

The tensile strength of the perforated template, which may have through-holes of a depth of at least 300 μm, allows for higher print pressures to be applied. By the term ‘print pressure’ we intend any force or pressure required to transfer the microstructure composition, using the squeegee blade for example, to the substrate. The print pressure applied may be determined by the printer to be used. Printers commonly used for this purpose may be from ASM (nee DEK) or ASYS. Some printers, for example, the 265 DEK Horizon, use mechanical pressure, whilst newer printers (from 2003 onwards), for example, the Horizon 01, allow for a feedback mechanism where the print pressure is increased until the pre-set print pressure is reached and contain sensors which are dynamic and therefore maintain the print pressure as it moves across the surface, such printers may apply a force of between 0 kg and 20 kg. Thus, the present invention provides a perforated template which can resist deformation above print pressures of at least 1 kg. For example, between 1 kg and 5 kg, between 1 kg and 10 kg, between 1 kg and 15 kg, between 1 kg and 20 kg, between 5 kg and 10 kg, between 5 kg and 15 kg, between 5 kg and 20 kg, between 10 kg and 15 kg, between 10 kg and 20 kg or between 15 kg and 20 kg. The inventors have surprisingly found that the use of perforated templates which are configured to resist deformation enables the production of strong microstructures without deformation.

In one embodiment of the present invention, the microstructures may be manufactured using a non-contact printing method, where the perforated template does not come in contact with the substrate the microstructure composition is to be deposited. Perforated templates with through-holes with depths less than 300 μm may result in template deformation when print pressures are higher than 1 kg, resulting in inconsistencies across the different microstructures. Surprisingly, the inventors have found that having a perforated template which has a rigidity configured to resist deformation results in a significant improvement over the prior art in terms of consistency and accuracy across different microstructures present on the same substrate.

In an alternate embodiment of the present invention, the microstructures may be manufactured using a contact printing method, where the perforated template is in contact with the substrate. In this instance, the inventors believe that the perforated template having through-holes of a depth of at least 300 μm allows for the protection of the microstructure as it is being created, resulting in the creation of more accurate and consistent microstructures at the end of the process compared to those present in the prior art. Additionally, having a thicker template in this method of printing allows for a higher deposit yield to be deposited, allows for the use of higher pressures (above 1 kg on the printing machines aforementioned), removes the possibility of any of the microstructure composition leaking underneath the perforated template and reduces the complexity of the process as no machine parameter changes are required, unlike the non-contact printing method.

It is envisaged that the method for manufacturing a microstructure according to the invention may further comprise the step of exposing the microstructure composition deposited on the substrate to a curing agent. The term ‘curing agent’ in the context of the present invention refers to an agent that is capable of facilitating the bonding of the molecular components of the microstructure composition in order to produce a hardened end product. In an embodiment, it is envisaged that the curing agent may be ultraviolet (UV) light.

Yet further, the method for manufacturing a microstructure according to the invention may further comprise one or more further steps comprising repeating the application of the microstructure composition, optionally wherein the perforated template is moved away from the substrate and re-positioned and aligned, such that the through-hole aligns with the microstructure, between each application of the microstructure composition. In an embodiment, it is envisaged that the template may be re-positioned using an alignment system of positional markings incorporated on the surface of the perforated template, and corresponding markers incorporated on the substrate onto which the microstructure composition is deposited. Using an alignment system of positional markings ensures that the perforated template can be re-positioned in an accurate manner relative to the portion of the microstructure already manufactured. A number of alignment systems may be suitable for this purpose, one such system is the DEK Hawkeye system.

The term ‘microstructure’ is intended to include any structure between 10 μm and 10 mm in height. For example, between 10 μm and 100 μm in height, between 10 μm and 500 μm in height, between 10 μm and 1000 μm in height, between 10 μm and 5000 μm in height, between 10 μm and 10000 μm, between 50 μm and 100 μm in height, between 50 μm and 500 μm in height, between 50 pm and 1000 μm in height, between 50 μm and 5000 μm in height, between 50 pm and 10000 μm, between 100 μm and 500 μm in height, between 100 μm and 1000 μm in height, between 100 μm and 5000 μm in height, between 100 μm and 10000 μm, between 500 μm and 1000 μm in height, between 500 μm and 5000 μm or between 500 μm and 10000 μm in height. Within the context of the present invention, the term ‘microstructure’ predominantly refers to structures located on transdermal patches. The microstructures may be microneedles and/or may take on various shapes or forms such as extended wires or doughnut shapes, for example.

It is envisaged that the microstructure disclosed herein may be a microneedle. By Thicroneedle' we intend a protrusion that will disrupt the stratum corneum and envisage that such microneedles may be between 10 μm and 1 mm in height. For example, between 10 μm and 100 μm in height, between 10 μm and 200 μm in height, between 10 μm and 300 μm in height, between 10 μm and 400 μm in height, between 10 μm and 500 μm in height, between 10 μm and 600 μm in height, between 10 μm and 700 μm in height, between 10 μm and 800 μm in height, between 10 μm and 900 μm in height, between 10 μm and 1000 μm in height. The presently disclosed method may also be applicable to a variety of different microstructures that would benefit from a novel technique in which accuracy, precision and scalability can all be achieved in an adequate time frame. Examples of microstructure shapes to which this method of manufacture could be applied may include doughnut shapes and extended wires. It is particularly envisaged that this method may be applied in the electrical and diagnostic fields where typically the components of such products are on a particularly small scale.

By ‘through-hole’ we intend an opening that extends completely through the material of an object, producing a continuous channel from one side of the object to the other.

It is envisaged that the perforated template and/or the through-hole may have a depth of 300 to 1000 μm. For example, the perforated template and/or through-hole may have a depth of 300 to 400 μm, 300 to 500 μm, 300 to 600 μm, 300 to 700 μm, 300 to 800 μm, 300 to 900 μm, 300 to 950 μm, 300 to 1000 μm, 350 to 400 μm, 350 to 500 μm, 350 to 600 μm, 350 to 700 μm, 350 to 800 μm, 350 to 900 μm, 350 to 950 μm, 350 to 1000 μm, 400 to 500 μm, 400 to 600 μm, 400 to 700 μm, 400 to 800 μm, 400 to 900 μm, 400 to 950 μm, 400 to 1000 μm, 450 to 500 μm, 450 to 600 μm, 450 to 700 μm, 450 to 800 μm, 450 to 900 μm, 450 to 950 μm, 450 to 1000 μm, 500 to 600 μm, 500 to 700 μm, 500 to 800 μm, 500 to 900 μm, 500 to 950 μm, 500 to 1000 μm, 550 to 600 μm, 550 to 700 μm, 550 to 800 μm, 550 to 900 μm, 550 to 950 μm, 550 to 1000 μm, 600 to 700 μm, 600 to 800 μm, 600 to 900 μm, 600 to 950 μm, 600 to 1000 μm, 650 to 700 μm, 650 to 800 μm, 650 to 900 μm, 650 to 950 μm, 650 to 1000 μm, 700 to 800 μm, 700 to 900 μm, 700 to 950 μm, 700 to 1000 μm, 750 to 800 μm, 750 to 900 μm, 750 to 950 μm, 750 to 1000 μm, 800 to 900 μm, 800 to 950 μm, 800 to 1000 μm, 850 to 900 μm, 850 to 950 μm, 850 to 1000 μm, 900 to 950 μm, 900 to 1000 μm or 950 to 1000 μm.

It is envisaged that the perforated template may be formed of plastic, stainless steel or nickel steel. The plastic may be acrylic, polypropylene, nylon, polyvinyl chloride (PVC) or polytetrafluorothylene (PTFE). These specific materials result in a number of desirable properties, including improved strength, flexibility, resilience, static properties and smoothness. It is understood that any other material also displaying these properties would be suitable for the present invention and that the aforementioned materials are commonly used in the electronic printed circuit industry and are desirable due to their inert characteristics.

It is envisaged that the microstructure composition may comprise a polymer. In an embodiment the microstructure polymer may be a UV-curable polymer, such that UV light is used to generate a crosslinked network of polymers. Examples of such UV-curable polymers include, but are not limited to, acrylate and methacrylate. However, a skilled person will recognise that any UV-curable polymer with the desired properties, for example, rapid solidification, would be appropriate for this purpose.

It is further envisaged that the microstructure composition may be self-adherent. The term ‘self-adherent’ is intended to refer to the ability of the microstructure composition to stick to itself without an additional element being introduced. This property enables a deposit of microstructure composition to be added to a preceding deposit, with each subsequent deposit adhering to the previous one, without the need for any additional components. The microstructure deposit may be the same or distinct from subsequent or preceding deposits, for example, in terms of the UV-curable polymer used.

It is envisaged that the through-hole of the perforated template may be substantially circular, square, rectangular, hexagonal, triangular or of a kidney bean shape. The shape of the microstructure used may be linked to the indication being treated. For example, some shapes may result in a larger surface area being covered compared to others. The shape of the microstructure or the array of microstructures may also be dependent on the body part to which it is intended to be applied. It is understood that the shape of the through-hole of the perforated template will be dependent on the shape and desired use of the microstructure in question.

It is envisaged that the through-hole of the perforated template may have a cross sectional width of between 50 and 600 μm, for example, 50 to 60 μm, 50 to 70 μm, 50 to 80 μm, 50 to 90 μm, 50 to 100 μm, 50 to 150 μm, 50 to 200 μm, 50 to 250 μm, 50 to 300 μm, 50 to 350 μm, 50 to 400 μm, 50 to 450 μm, 50 to 500 μm, 50 to 550 μm, 50 to 600 μm, 60 to 70 μm, 60 to 80 μm, 60 to 90 μm, 60 to 100 μm, 60 to 150 μm, 60 to 200 μm, 60 to 250 μm, 60 to 300 μm, 60 to 350 μm, 60 to 400 μm, 60 to 450 μm, 60 to 500 μm, 60 to 550 μm, 60 to 600 μm, 70 to 80 μm, 70 to 90 μm, 70 to 100 μm, 70 to 150 μm, 70 to 200 μm, 70 to 250 μm, 70 to 300 μm, 70 to 350 μm, 70 to 400 μm, 70 to 450 μm, 70 to 500 μm, 70 to 550 μm, 70 to 600 μm, 80 to 90 μm, 80 to 100 μm, 80 to 150 μm, 80 to 200 μm, 80 to 250 μm, 80 to 300 μm, 80 to 350 μm, 80 to 400 μm, 80 to 450 μm, 80 to 500 μm, 80 to 550 μm, 80 to 600 μm, 90 to 100 μm, 90 to 150 μm, 90 to 200 μm, 90 to 250 μm, 90 to 300 μm, 90 to 350 μm, 90 to 400 μm, 90 to 450 μm, 90 to 500 μm, 90 to 550 μm, 90 to 600 μm, 100 to 150 μm, 100 to 200 μm, 100 to 250 μm, 100 to 300 μm, 100 to 350 μm, 100 to 400 μm, 100 to 450 μm, 100 to 500 μm, 100 to 550 μm, 100 to 600 μm, 150 to 200 μm, 150 to 250 μm, 150 to 300 μm, 150 to 350 μm, 150 to 400 μm, 150 to 450 μm, 150 to 500 μm, 150 to 550 μm, 150 to 600 μm, 200 to 250 μm, 200 to 300 μm, 200 to 350 μm, 200 to 400 μm, 200 to 450 μm, 200 to 500 μm, 200 to 550 μm, 200 to 600 μm, 250 to 300 μm, 250 to 350 μm, 250 to 400 μm, 250 to 450 μm, 250 to 500 μm, 250 to 550 μm, 250 to 600 μm, 300 to 350 μm, 300 to 400 μm, 300 to 450 μm, 300 to 500 μm, 300 to 550 μm, 300 to 600 μm, 350 to 450 μm, 350 to 500 μm, 350 to 550 μm, 350 to 600 μm, 400 to 450 μm, 400 to 500 μm, 400 to 550 μm, 400 to 600 μm, 450 to 500 μm, 450 to 550 μm, 450 to 600 μm, 500 to 550 μm, 500 to 600 μm or 550 to 600 μm. It is understood that the through-holes of the perforated template may be larger than the microstructure to be manufactured. In some instances, the through-hole may be larger than a section of the microstructure, therefore resulting in the microstructure composition being applied to a specific section. The diameter of the through-holes of the perforated template will therefore depend on the given diameter of the microstructure to be manufactured.

It is envisaged that the through-hole of the perforated template may be formed by electroforming, laser-drilling or a convention drill bit. Electroforming in the context of this invention refers to a process by which electrodeposition occurs; the deposition of metal onto a conductive object. For this process it is necessary to have a power source, two electrodes (an anode and a cathode) and a solution of metallic salts within an electrolytic bath. The result is that metallic ions are converted into atoms which build up onto the cathode surface through a continuous deposit. Using this method it is possible to control both the thickness and shape of the end product. Laser-drilling refers to the process whereby a through-hole is created by repeatedly pulsing focused energy on a material, such as pulsed laser energy emitted from a solid-state laser. Using this method, through-holes with very small diameters and precisely engineered shapes can be produced. Larger through-holes can also be produced by moving the laser around the circumference of the originally made through-hole until the desired diameter is achieved. Alternatively, the through-hole may be created by using a conventional drill bit. Through-holes of different sizes may therefore be created by using different sized drill bits. A skilled person would understand that other suitable methods available in the art may alternatively be used for creating through-holes according to the invention.

It is envisaged that the perforated template may be positioned within a supporting frame such that the perforated template is maintained in the correct position. The supporting frame may be connected physically to the template via another material and non-interchangeable. Alternatively, the frame may accommodate switching of templates by using a method of clamping the template into position using a system such as DEK VectorGuard or fasteners.

The microstructure may be one of an array of microstructures to be manufactured using the present invention herein described. By ‘array’ we intend multiple microstructures present on a single solid substrate which display a specific arrangement relative to each other. For example, an array of microstructures may be a transdermal patch with a plurality of microstructures and positioned in a uniform manner across the supporting solid substrate. A skilled person in the art will recognise that different patterns of microstructures may be utilised to enhance delivery of the desired compound depending on the body part to be treated.

It is envisaged that the substrate may be a PVC substrate, a metal substrate, a poly-lactic acid substrate, a glass substrate, a ceramic substrate, a polystyrene substrate, a cellulose based substrate, a poly-vinyl alcohol substrate, a polycarbonate substrate, a poly-methyl methacrylate substrate, a silicone substrate, a poly-ethylene terephthalate substrate, a polyurethane substrate or a nitrocellulose substrate. It is understood that the substrate may be formed from any material suitable for contact with human, or non-human, exterior surfaces and displaying the desired properties, for example, flexibility and durability. Examples of such exterior surfaces include skin, mucous membranes, the oral cavity or eyes of the subject. In an embodiment the substrate may form at least a part of a transdermal patch.

It is envisaged that the method of manufacturing a microstructure according to the present invention may comprise a further step of applying a coating composition to the microstructure. In an embodiment, the coating composition may comprise an active ingredient, an active pharmaceutical ingredient, and electrically conductive material and/or a bio-chemical reagent. Alternatively, the coating composition may form a structural protective barrier over the microstructure.

The term ‘active ingredient’ is taken to include active ingredients of both a pharmaceutical and non-pharmaceutical nature. Non-pharmaceutical active ingredients may be used, for example, in the cosmetic industry. Examples of active ingredients which are non-pharmaceutical in nature include alpha-hydroxy acids, beta-hydroxy acid, hydroquinone, kojic acid, retinol, L-ascorbic acid, hyaluronic acid, copper peptide, alpha-lipoic acid and dimethylaminoethanol. The coating composition in this instance may be dissolvable, allowing for the release of the active ingredient at the desired time. The active ingredient may be released from the coating composition either by the coating itself dissolving or via a diffusion gradient. The coating may be triggered to dissolve by using a primer to provide a sticky surface which may be engineered to dissolve and release the active ingredient on contact with water in the skin or via a temperature change, for example, its melting point. In some instances, a primer step may not be required. The active ingredient within the coating composition may be delivered at either the surface of the skin or delivered much deeper into the dermis. The extent of delivery will be dependent on the length of the microstructure and the indication to be treated. An active ingredient within the context of the present invention refers to the element of the coating composition which is biologically active and thus produces the desired therapeutic effect.

If the active ingredient is an active pharmaceutical ingredient, the active pharmaceutical ingredient may be a biologically active skin regenerative compound, preferably wherein the skin regenerative compound is hyaluronic acid, vitamin B, vitamin C, co-enzyme Q10, matrixyl or resveratrol. It is intended that these skin regenerative compounds may be utilised to manufacture cosmetic products comprising microstructures coated using the methods described herein. Such products may be used to treat aesthetic problems for patients. Such conditions to be treated may include lateral canthal rhytids (crow-feet), perioral dermatitis, tear trough deformity, décolletage wrinkles (chest wrinkles) and age spots. Additionally, skin regenerative compounds may include compounds aimed at improving the appearance of scarring or to encourage the speed of wound healing. A person skilled in the art will recognise that the compound to be included in the coating composition will depend on the condition to be treated. For example, hyaluronic acid may be a more suitable compound to use for lateral canthal rhytids than vitamin B or C. Additionally, the coating composition may comprise just a single skin regenerative compound or may include a combination of compounds, where two or more different compounds are present within the same coating composition. The latter may be particularly useful if treating patients with multiple conditions to be addressed.

Further, the active pharmaceutical ingredient may be an analgesic, an anti-inflammatory and/or an immunosuppressant compound, preferably wherein the compound is diclofenac, ibuprofen, lidocaine or hydrocortisone. An analgesic is taken to be a compound whose primary action is to relieve pain in a subject. An anti-inflammatory compound is taken to be a compound that can relieve inflammation associated with a particular injury or condition. An immunosuppressant compound is taken to be a compound that inhibits or prevents the activity of the immune system. It is intended that the above compounds may be suitable for use as a method of pain control, whether it is used as general pain relief or as a local anaesthetic, or for dermatological conditions such as psoriasis or eczema. A person skilled in the art would recognise that the above listed compounds may fall into more than one of the above groups. For example, ibuprofen is recognised to be both an analgesic and an anti-inflammatory. It is understood that the compound to be included in the coating composition will depend on the condition to be treated. For example, hydrocortisone will be more suitable for treating eczema than ibuprofen. Additionally, the coating composition may comprise just a single analgesic, anti-inflammatory or immunosuppressant compound or may include a combination of compounds, where two or more different compounds are present within the same coating composition. The latter may be particularly useful if treating patients with multiple conditions to be addressed.

The active ingredient may be released over a timescale of seconds to hours. The coating compositions may be tailored to have various release times according to the end application, for example, either an acute or chronic condition to be treated.

In addition to the relevant active ingredient for the condition in question, the coating composition may further comprise compounds suitable for use in a pharmaceutical formulation. Such compounds may include acceptable pharmaceutical carriers, examples of which include encapsulation vehicles, nano-particles, micelles, skin penetration enhancers and other excipients.

The coating composition may comprise an electrically conductive material to aid the movement of active ingredients across the stratum corneum. In this instance, an externally placed electrode would be required to deliver physiologically acceptable electrical currents (0.1-1 mA/per cm²). Examples of such an electrically conductive material may be the addition of conductive polymers to the coating composition; organic polymers that are known to conduct electricity. Conductive polymers may include polyacetylene, polypyrrole, polyindole and polyanailine to highlight a few. It is understood that any electrically conductive material appropriate for contact with the human body may be appropriate for inclusion in the coating composition.

The coating composition may comprise a bio-chemical reagent. By the term tio-chemical reagent' we intend any biological material or organic compound that can be found in a biological system, or that can be used for biological research. For example, the coating composition may comprise nucleotides and/or amino acids intended for therapeutic or cosmetic use, for example, enhancing collagen production. The inclusion of a bio-chemical reagent may also aid in diagnostic processes.

Additionally, the coating composition may form a structural protective barrier over the microstructure. This may help protect a more fragile microstructure from breaking as it moves through the outer most layer of the skin; the stratum corneum. The protective barrier may cover the entirety of the microstructure or just part of the microstructure, for example, the tip as the most delicate part. A structural protective barrier coating may require at least a single coating. The structural protective barrier formed by the coating composition may be triggered to dissolve by the exposure of the coating composition to a gas or a liquid, such as a solvent, for example, an alcohol in liquid or vapour phase, resulting in the exposure of the microstructure.

It is envisaged that a pre-coating layer may be applied to the perforated template prior to the perforated template being in contact with the microstructure composition. This may improve the accuracy and consistency of the resulting microstructures by providing a frictionless surface or by reducing the surface tension on top of which the microstructure composition can be applied. Examples of such coatings may include polytetrafluoroethylene (PTFE), perfluoroalkoxy alkane (PFA) and fluorinated ethylene propylene (FEP). The chosen substance for pre-coating the perforated template will ideally be inert and leave no residue on the microstructure being built, or the substrate being built upon, to prevent any unwanted additive effect that may not be compatible for medical use.

Accordingly, in a second aspect, the present invention provides for a transdermal patch comprising the microstructure(s) manufactured by the method herein defined. It is expected that the patch will of course comprise multiple microstructures arranged as appropriate for the intended application of the patch. It is envisaged that the transdermal patch comprising one or more microstructures may aid in the delivery of an active substance to a subject or be used in the diagnosis of a condition, for example, a bacterial or viral infection. It is further envisaged that the transdermal patch herein described may be used as a device for monitoring the success of a variety of treatments, for example, chemotherapy or antibiotic success rates.

The present invention may also provide a kit comprising the transdermal patch herein described and instructions for use of the transdermal patch. The instructions may indicate how long the patch should be applied and in what matter and/or how often the patch, or multiple patches, should be re-applied as appropriate. The kit could accommodate patches with different dosage or different active pharmaceutical ingredients to be applied as instructed by a physician.

The present invention may also provide for a therapeutic or cosmetic method of treating a condition in a patient in need thereof comprising applying the transdermal patch herein described to the subject's skin.

It is envisaged that patches manufactured and coated using the method provided herein and patches provided herein may be useful in treating conditions such as lateral canthal rhytids (crow-feet), perioral dermatitis, tear trough deformity, décolletage wrinkles (chest wrinkles), age spots, stretch marks, scarring, hair loss, psoriasis, eczema and/or dry skin. Further, conditions to be treated using methods provided herein could include pain and/or inflammation.

The extent of penetration of the microstructures into the skin may be dependent on the skin condition. For example, coarse skin may require longer microstructures to break through the skin, whilst damaged skin may require penetration to a lesser depth to achieve the same effect.

It is envisaged that the present invention may also be applicable for veterinary applications. It is expected that in order for the transdermal patch to be effective, the animal skin may have to be shaved off to enable direct contact with the skin. The term ‘animal’ includes all vertebrate mammals, such as non-human primates, sheep, dogs, cats, horses, goats, cows, and chickens.

The invention will now be illustrated in the following examples with reference to the accompanying drawings.

### EXAMPLES

The inventors of the present invention have discovered that the use of a deep template, or a perforated template which can resist deformation, results in a surprising reduction in print passes compared to using a thinner template and results in a higher deposit yield of microstructure composition whilst maintaining the height of the microstructure (Table 1). As a result of this finding the inventors have been able to increase the print image area from an area of 15×15 cm to 50×50 cm. Additionally, the use of a thicker template results in the ability to use higher pressures when using a squeegee blade to control movement of the microstructure composition across the template without the template flexing and causing a number of disadvantages. Accordingly, the present invention solves a number of problems associated with previously used methods. For example, the present invention allows for 100% contact printing, protection of the microstructure whilst being built, unwanted flexing of the template, a significant reduction in the amount of microstructure composition to be cured which sticks to the template, and a reduction in the number of printing passes needed to produce the end product. The present invention is therefore a more efficient, more accurate and more cost-effective method than those methods already known in the art.

TABLE 1
Comparison of the properties of microneedles
with varying template thickness.

| | | | Microstructure |
|---|---|---|---|
| Template | Print | Microstructure | Composition |
| Thickness μm | Passes | Height μm | Deposit Yield μm |
| 100 | 22 | 530 | 24 |
| 200 | 16 | 480 | 30 |
| 250 | 12 | 460 | 38 |
| 500 | 5 | 520 | 104 |

1. A method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template and/or the through-hole have a depth of at least 300 μm.

2. A method for manufacturing a microstructure, the method comprising the step of applying a microstructure composition to a perforated template comprising through-holes, wherein the microstructure composition passes through a through-hole and is deposited on a substrate, thereby forming a microstructure, characterised in that the perforated template has a rigidity configured to resist deformation.

3. The method of claim 1, wherein the perforated template and/or through-hole has a rigidity configured to resist deformation.

4. The method of claim 2, wherein the perforated template resists deformation of a print pressure of at least 1 kg to 20 kg.

5. The method of claim 2, wherein the perforated template and/or through-hole have a depth of at least 300 μm.

6. The method of claim 1, further comprising the step of exposing the microstructure composition deposited on the substrate to a curing agent, preferably wherein the curing agent is ultraviolet (UV) light.

7. The method of claim 1, wherein the method further comprises one or more further steps comprising repeating application of the microstructure composition, optionally wherein the perforated template is moved away from the substrate and re-positioned and aligned, such that the through hole aligns with the microstructure, between each application of the microstructure composition.

8. The method of claim 7, wherein the perforated template is re-positioned using an alignment system of positional markings incorporated on a surface of the perforated template, and corresponding markers incorporated on the substrate onto which the microstructure composition is deposited.

9. The method of claim 1, wherein the perforated template and/or the through-hole have a depth of 300 to 1000 μm.

10. The method of claim 1, wherein the perforated template is formed of plastic, stainless steel or nickel steel.

11. The method of claim 1, wherein the microstructure composition comprises a polymer, preferably wherein the microstructure composition is a UV-curable polymer.

12. The method of claim 1, wherein the through-hole of the perforated template is substantially circular, square, rectangular, hexagonal, triangular or of a kidney bean shape.

13. The method of claim 1, wherein the through-hole of the perforated template has a cross-sectional width of between 50 and 600 μm.

14. The method of claim 1, wherein the through-hole of the perforated template is formed by electroforming, laser-drilling or a conventional drill bit.

15. The method of claim 1, wherein the perforated template is positioned within a supporting frame such that the perforated template is maintained in the correct position.

16. The method of claim 1, wherein the microstructure is one of an array of microstructures.

17. The method of claim 1, wherein the microstructure is a microneedle.

18. The method of claim 1, wherein the substrate is a PVC substrate, a metal substrate, a poly-lactic acid substrate, a glass substrate, a ceramic substrate, a polystyrene substrate, a cellulose based substrate, a poly-vinyl alcohol substrate, a polycarbonate substrate, a poly-methyl methacrylate substrate, a silicone substrate, a poly-ethylene terephthalate substrate, a polyurethane substrate or a nitrocellulose substrate.

19. The method of claim 1, wherein the substrate forms at least a part of a transdermal patch.

20. The method of claim 1, wherein the method comprises a further step of applying a coating composition to the microstructure.

21. The method according to claim 20, wherein the coating composition comprises an active ingredient, an active pharmaceutical ingredient, an electrically conductive material and/or a bio-chemical reagent.

22. A transdermal patch comprising a microstructure manufactured using the method of claim 1.

23. A kit comprising a transdermal patch according to claim 22 and instructions for use of the transdermal patch.

24. A therapeutic or cosmetic method of treating a condition in a patient in need thereof comprising applying the transdermal patch according to claim 22 to the patient's skin.

25. The method according to claim 24, wherein the condition is lateral canthal rhytids (crow-feet), perioral dermatitis, tear trough deformity, décolletage wrinkles (chest wrinkles), age spots, stretch marks, scarring, hair loss, psoriasis, eczema and/or dry skin, or wherein the condition is pain and/or inflammation.
